(19) United States
(12) Patent Application Publication
(10) Pub. No.: US 2017/0267957 A1
(43) Pub. Date: Sep. 21, 2017

(54) PHOTOBIOREACTOR

(71) Applicant: AGRONOSIS PTE LTD, Singapore (SG)

(72) Inventor: Wai Man LAU, Singapore (SG)

(73) Assignee: AGRONOSIS PTE LTD, Singapore (SG)

(21) Appl. No.: 15/532,176

(22) PCT Filed: Oct. 19, 2015
(86) PCT No.: PCT/SG2015/050398

§ 371 Date: Jun. 1, 2017

(30) Foreign Application Priority Data
Dec. 2, 2014 (SG) 10201408033V
Apr. 3, 2015 (CN) 201520201888.5

(51) Int. Cl. C12M 1/00; C12M 3/00; C12M 1/34; C12N 1/12; C12M 1/12

(52) U.S. Cl. CPC C12M 21/02; C12M 1/00; C12M 23/58; C12N 1/12; C12M 21/12; C12M 23/04; C12M 23/34; C12M 41/12; C12M 3/00; A01G 7/00

(57) ABSTRACT

A photobioreactor for cultivating photoautotrophs that comprises a first hatcher for containing the photoautotrophs and a first feed medium; a second hatcher for holding the photoautotrophs and a second feed medium; and a pump connected to the first hatcher, the second hatcher or both the first hatcher and the second hatcher for moving the photoautotrophs between the first hatcher and the second hatcher or vice versa.

The present application relates to a photobioreactor (PBR) for growing photoautotrophs, microalgae or microphytes. Both photoautotrophs and microalgae may simply be known as algae. The photobioreactor is also known as a hatcher or incubator. The application further relates methods of making, assembling, disassembling, installing, configuring, maintaining and using the photobioreactor.

The present application claims the priority date of the earlier Singapore patent application “Photobioreactor”, which has the serial number of 10201408033V and the filing date of 2 Dec. 2014. The present application further claims the priority date of the earlier Chinese Utility Model application with the same title, which has the serial number of 201520201888.5 and ZL 2015 2 0201888.5 with the filing date of 3 Apr. 2015. All content and/or subject matter of these priority applications is/are hereby incorporated entirely.

Algae have chlorophyll as their primary photosynthetic pigment and lack a sterile covering of cells around their reproductive cells. Algae have photosynthetic machinery ultimately derived from cyanobacteria that produce oxygen as a by-product of photosynthesis. Since algae can be utilised as an energy source, fertiliser, nutrition supplement, pollution control medium (e.g. carbon dioxide reduction), pigments and substance stabiliser (e.g. carrageenan), many scientists, research institutions and corporations have investigated and proposed diverse types of photobioreactors and hatcheries for cultivating algae with large volumes. Productive, cost effective and robust photobioreactors are desirable for industrial implementation in many parts of the world in order to deal with the current energy and environmental crisis.

The present application aims to provide a new and useful photobioreactor for growing photoautotroph, microalgae or algae. The application also aims to offer new and useful methods for growing the photoautotroph, microalgae or algae. Essential features of the relevant invention(s) are provided by independent claims, whilst important features of the invention(s) are presented by their dependent claims.

According to a first aspect, the application provides a photobioreactor that comprises a first hatcher for containing a first feed medium of the photoautotroph, the first hatcher having an inlet and outlet; a second hatcher for holding a second feed medium of the photoautotroph, the second hatcher also having an inlet and outlet; and a valve connected the outlet of the first hatcher to the inlet of the second hatcher for regulating flow of the feed media and/or photoautotroph between the first hatcher and the second hatcher. Hence, the photoautotroph, microalgae or algae can be cultivated in separate photobioreactors such that the photoautotroph, microalgae or algae can be grown in different controlled environments or altered conditions. Since the photoautotroph, microalgae or algae has a few stages of growth, which can be optimised with different aqueous cultures (i.e. algaculture), the multiple hatcheries offer isolated and controlled situations that suit growth of the photoautotroph at different stages, thus optimising production volume and quality of the photoautotroph. For example, light exposure, temperature, flow speed, nutrient content, salinity and trace elements' concentration can be made different at the multiple photobioreactors. These environmental factors (e.g. temperature, light exposure) can further be continuously, periodically or intermittently monitored or regulated.

Additionally, since the two hatcheries are connected by the valve, contents of the two hatcheries can be transferred between each other. For example, when fourteen or fifteen hatcheries are serially connected, the photoautotroph, microalgae or algae can be progressively pushed through these hatcheries so that each of the hatcheries provides a suitable growth environment to various growth stages of the photoautotroph, microalgae or algae. The photoautotroph, microalgae or algae from one or more of these hatcheries can be harvested, discharged or processed when necessary. The photoautotroph, microalgae or algae from one of these hatcheries or aqueous culture can be individually treated and returned back to any of the hatcheries.

The valve regulates, directs or controls the flow of a fluid (gases, liquids, fluidized solids, or slurries) by opening, closing, or partially obstructing various passageways. For example, the valve includes a check valve that allows the photoautotroph and the aqueous culture to flow from the first hatchery to the second hatchery only, not vice versa. Alternatively, the valve comprises a narrow tube that can keep flow rate of the photoautotroph and the aqueous culture to be lower than a predetermined value (e.g. 02 litre per hour). On the other hand, the valve has a pool that can delay or withhold transfer of the photoautotroph and the aqueous culture. Alternatively, the valve serves as a fluid passage without restricting fluid communication during normal operation. Accordingly, any of these multiple hatcheries can be independently operated when necessary, or collectively utilised when required, providing much flexibility to the photobioreactor. In other words, one or connectors (e.g. joints, couplings, junctions, bridges, passages) that link the multiple hatcheries with different growth environments can be considered as the valve(s).

The photobioreactor provides productive and cost effective facilities to cultivate algae commercially and/or industrially, which offers food ingredients such as omega-3 fatty acids or natural food colorants and dyes, food, fertilizer, bioplastics, chemical feedstock (raw material), pharmaceuticals, algal biofuel and means of pollution control. The photobioreactor can operate in “batch mode”, which involves restocking the hatcheries or the photobioreactor after each harvest, but the photobioreactor is also possible to grow and harvest continuously. Continuous operation of the photobioreactor provides precise control of all elements with optimised productivity so that the photobioreactor provides sterilized water, nutrients, air, and carbon dioxide at the correct rates, which allows the photobioreactor to operate for prolonged periods. The “log phase” production of microalgae or algae generally offers higher nutrient content than “senescent” algae. On or more parts of the photobioreactor (e.g. container, hatchery) is possibly made by transparent or translucent materials so that the microalgae in the photobioreactor can perform photosynthesis when sealed inside.

The photobioreactor may further comprise a third hatcher that is sequentially or in parallel connected from the first hatcher to the second hatcher for growing the photoautotrophs (i.e. photoautotroph) progressively in the two or three hatchers with different feed media. According to the serial connection scheme, nutrients level, salinity and/or trace element content may be progressively escalated from the first hatchery, the second hatchery to the third hatchery for adjusting or increasing salinity, nutrient concentration level or both from the first hatcher, the second hatcher, the third hatcher, optionally another hatcher and the final hatcher gradually. Some of the multiple hatcheries may be connected serially (e.g. as an endless loop) or in parallel.

One or more of the hatcheries can comprise a feeder that is connected to the first hatcher, the second hatcher, the third hatcher or a combination of any of these hatcheries or any part of the photobioreactor for providing a feed medium to the photoautotroph. The feeder can keep a predetermined amount of feedstock for supplying to one or more hatcheries. The feeder can additionally process and formulate the feedstock for providing suitable feedstocks to the hatcheries. Accordingly, the feeder can monitor and/or regulate the feedstock manually or automatically (e.g. by computer) so that stable and efficient production of the photoautotroph.

The feeder may comprise a first feeder connected to the first hatcher for providing the first feed medium. The feeder may further comprise a second feeder connected to the second hatcher for providing the second feed medium. The feeder may further comprise a third feeder connected to the third hatcher for providing the third feed medium. Hence, each or several hatcheries can be separately supplied and/or regulated with their feedstock. For example, two of the hatcheries may be connected to the same feeder, whilst the same feeder may provide the valve for supplying different amount of feedstocks to the two of the hatcheries.

The photobioreactor can additionally comprise a final hatcher further directly or indirectly connected to the third hatcher for containing a final feed medium of the photoautotroph; and a final feeder that is connected to the final hatcher for providing the final feed medium. The final hatcher is a last-stage incubator for growing the photoautotroph, microalgae or algae. The final hatcher, the final feeder or both can be integrated together, similar or different from other hatchers and feeders.

The photobioreactor may further comprise one or more harvest channels that is connected to any part or any outlet of the photobioreactor for harvesting matured photoautotroph. The harvest channel is alternatively known as a harvester for dewatering and drying the matured photoautotroph. On the other hand, the microalgae may be harvested using micro-screens, by centrifugation, by flocculation and by froth flotation, which are also known as the harvest channel or harvester. For example, the harvester includes two belts moving in opposing directions such that desired photoautotroph solid is poured through a spout on to a top belt. Water passes through the belt, while the solid remains on top. Alternatively, multiple harvest channels may be connected to any of the hatcheries.

The photobioreactor can further comprise one or more pumps connected to the first hatcher, the second hatcher, the third hatcher, the final hatcher, a combination of these hatcheries or any part of the photobioreactor for moving the photoautotrophs the feed media, or both the photoautotrophs and the feed media. The pump includes direct lift type, displacement type, and gravity type pumps, such as paddle wheels. When electrically powered, the pump can be conveniently operated (e.g. on/off) for regulating flow rate of relevant fluids or slurries. The pump can further agitate the photoautotroph with or without compressed air for lifting photoautotroph from lower regions of the hatcheries and preventing over-exposure to the sun or artificial light.

One or more of the photobioreactors may comprise a gas-liquid distributor at a bottom or a side of the one or more photobioreactors for circulating liquid or gas in the one or more photobioreactors. The gas-liquid distributor may manage odour, oxygen and/carbon dioxide at the respective hatchery so that neither eutrophication nor hypoxia is likely to occur. The gas-liquid distributor may allow re-distribution of gas and liquid as well as the microalgae cells to achieve a specific circulation pattern ideal for each of the hatcheries. The size of the gas-liquid distributor opening may be sufficiently large to prevent clogging of the gas-liquid distributor and not causing shear damage to the microalgae cells.

The one or more hatcheries or the first hatcher and the second hatcher can be vertically stacked. Since the first hatcher can be positioned at the bottom of the photobioreactor, stirring of the aqueous culture and microalgae can be achieved by gas-liquid movement through the gas-liquid distributor and/or action of the pump at lower portions of the hatcheries or photobioreactor.

Two or more of the hatcheries (e.g. the first hatcher and the second hatcher) may be horizontally laid out on substantially the same level or horizontally aligned. Less energy is required to stir up content of the hatcheries as compared to that of the vertical alignment. If land areas or waterbody (sea, lake, pond or river) surfaces are available at low cost, the horizontal alignment provides convenience for access when positioned at about a table height (e.g. about 70 centimetres).

Two or more of the hatcheries (e.g. first hatcher, the second hatcher, the third hatcher and the final hatcher) can be different in profiles, shapes, sizes, volume, pressure, light exposure, structure, temperature or other properties for growing the photoautotrophs differently (i.e. under different conditions or environments). For example, the first hatcher utilises sunlight with full spectrum of visible light, whilst the second hatcher is exposed to artificial light by LED (light-emitting diode) light bulbs a few hours per day. Tailored hatcheries or growth environment can additionally facilitate the management of the algae growth.

The photobioreactor may further comprise one or more sensors for monitoring nutrients in the feed media, growth condition of the photoautotrophs, components or growth conditions of the photobioreactor. The one or sensors may include diverse types of sensors relating to acoustic, sound, vibration, automotive, transportation, chemical, electric current, electric potential, magnetic, radio, flow, fluid velocity, ionizing radiation, subatomic particles, navigation instruments, position, angle, displacement, distance, speed, acceleration, optical, light, imaging, photon, pressure, force, density, level, thermal, heat, temperature, proximity and presence. The one or more sensors may be connected to data logger or computers wirelessly or wires/cables so that the photobioreactor may be operated remotely, automatically or manually. The one or more sensors may be integrated with sensor networks so that multiple photobioreactors and factories of the photoautotrophs may be collectively operated or monitored. Control factors (e.g. temperature, nutrients concentration levels) may be automatically adjusted in response to both internal (e.g. size of the photoautotrophs) and external factors (sunlight conditions).

The one or more sensors can comprise one or more chemical sensors for monitoring chemical composition of the feed media. The chemical sensor is a self-contained analytical device that can provide information about chemical composition of the feedstock or aqueous culture a liquid or a gas phase. Data collected by the sensor is provided in the form of a measurable physical signal that is correlated with the concentration of a certain chemical species (termed as analyte). For example, the chemical sensor is a pH sensor (pH meter) that assist to examine and control pH values in one of the hatcheries.

The one or more sensors may comprise a biosensor for observing growth of the photoautotroph. The biosensor combines a biological component with a physicochemical detector. The biosensor may be connected to a biosensor reader device with the associated electronics or signal processors (e.g. the computer) that are primarily responsible for the display of the results in a user-friendly way. For example, the biosensor may be used to measure nutrient concentration percentage and bacteria levels.

The present application additionally provides a cluster of photobioreactors, which comprises the photobioreactor and an open pond photobioreactor, a tube photobioreactor, a closed reactor (photobioreactor), a horizontal photobioreactor, a vertical photobioreactor, a flat plate reactor, a fermentor-type reactor or a combination of any of these (that is/are) connected to the photobioreactor. In other words, several photobioreactors of the same or different types (e.g. open systems, close systems) can be connected for growing diverse types of microalgae collectively or separately.

According to a second aspect, the present application provides a method for cultivating photoautotroph that comprises a first step of providing a first hatcher for cultivating photoautotrophs with a first feed medium; a second step of offering a second hatcher for growing the photoautotrophs with a second feed medium; and a third step of moving a portion of the photoautotroph, the first feed medium, the second feed medium or a combination of any of these between the first hatcher and the second hatcher for regulating (e.g. enhancing or reducing) growth of the photoautotroph. Some of these steps may be changed in sequence or combined. The method presents discrete compartments and/or method stages for providing suitable or optimised environments to enhance photosynthesis. The proposed method will greatly enhance production quality, quantity and efficiency for growing microalgae.

The method may further a step of harvesting the photoautotrophs from the second hatcher that is connected to the first hatcher for progressively growing the photoautotroph. Although harvesting or extracting photoautotrophs may performed at any stage of photoautotrophs growth, the harvesting at a later or final stage of the photobioreactor facilitates the collection of most matured or excellent quality of microalgae (i.e. microphytes) such that carotenoids, antioxidants, fatty acids, enzymes, polymers, peptides, toxins and sterols may be produced with lower cost, higher quality and more quantity.

The method can further comprise a step of propelling the photoautotrophs, the aqueous cultures or feedstocks from the first hatcher for growing the photoautotrophs in the first medium to the second hatcher in order to cultivate the photoautotrophs in different media. Transferring of the photoautotrophs, the aqueous cultures or feedstocks help to reduce or eliminate physical constrain of the photoautotrophs so that the photoautotrophs can be grown flexibly.

The method may further comprise a step of adjusting salinity, temperature, light exposure, nutrient concentration level, oxygen content, carbon dioxide volume, fluid flow rate or other growth environmental factors in at least one of the hatcheries. Hence, growth environment or conditions at the hatcheries or photobioreactor may be fine-tuned and adjusted in real-time. The photobioreactor thus become more robust for dealing external (e.g. change of sunlight) or internal (discharge of odour) instability.

The method can further comprise a step of sensing operating condition of the hatcheries. The sensing or monitoring can be carried out manually by operators or automatically by sensors connected to the computer. The photobioreactor accordingly become more responsive to growth conditions of the microscopic algae, making the photobioreactor highly productive and easily operated by non-experts.

The method may additionally comprise a step of collecting growth data of the photoautotrophs via a sensor network via a computer. Off-site or remote scientists or engineers may offer assistance, control or trouble-shoot without incurring cost of physical travelling. Moreover, researchers and manufacturers may collaborate by sharing the growth data. The computer includes a portable electronic device (e.g. mobile phone), a computing server, a personal computer a mainframe computer or a combination of any of these.

Alternatively speaking, the present application provides strategy of offering mixed environment cultivation in hatcheries such that growth of microalgae has been enhanced significantly. The hatcheries or hatcher is also known as photobioreactor(s). Most of these microalgae species produce unique products like carotenoids, antioxidants, fatty acids, enzymes, polymers, peptides, toxins and sterols.

The accompanying figures (Figs.) illustrate embodiments and serve to explain principles of the disclosed embodiments. It is to be understood, however, that these figures are presented for purposes of illustration only, and not for defining limits of relevant inventions.

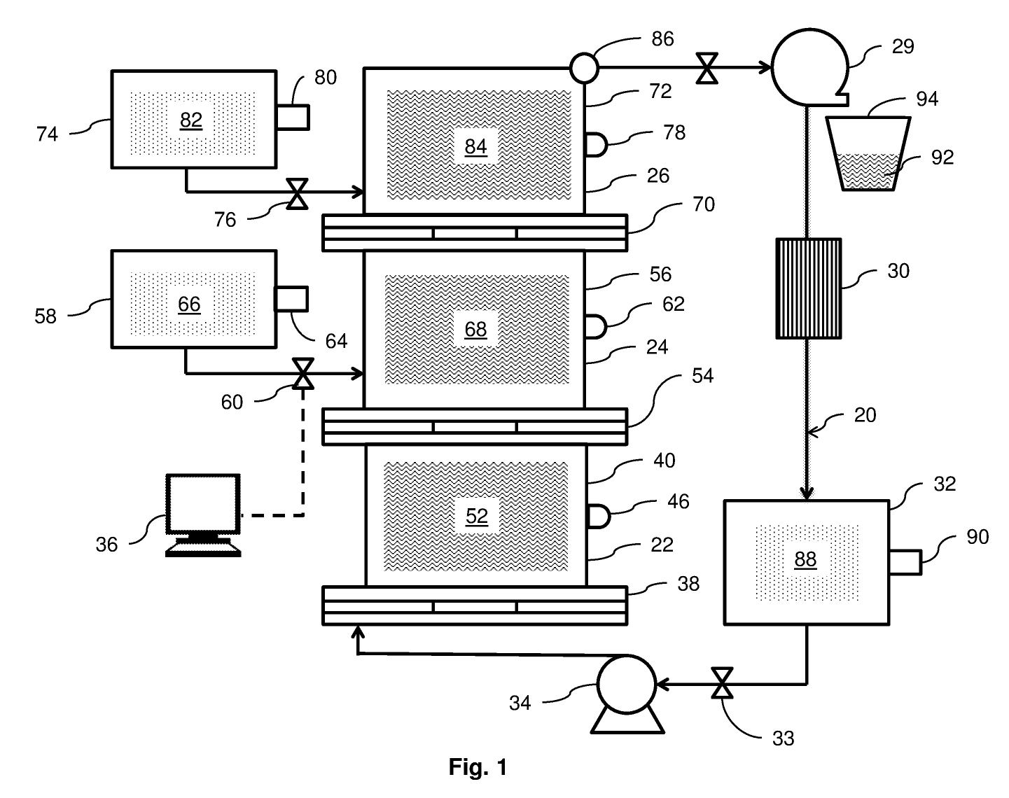

FIG. 1 illustrates a first photobioreactor; and

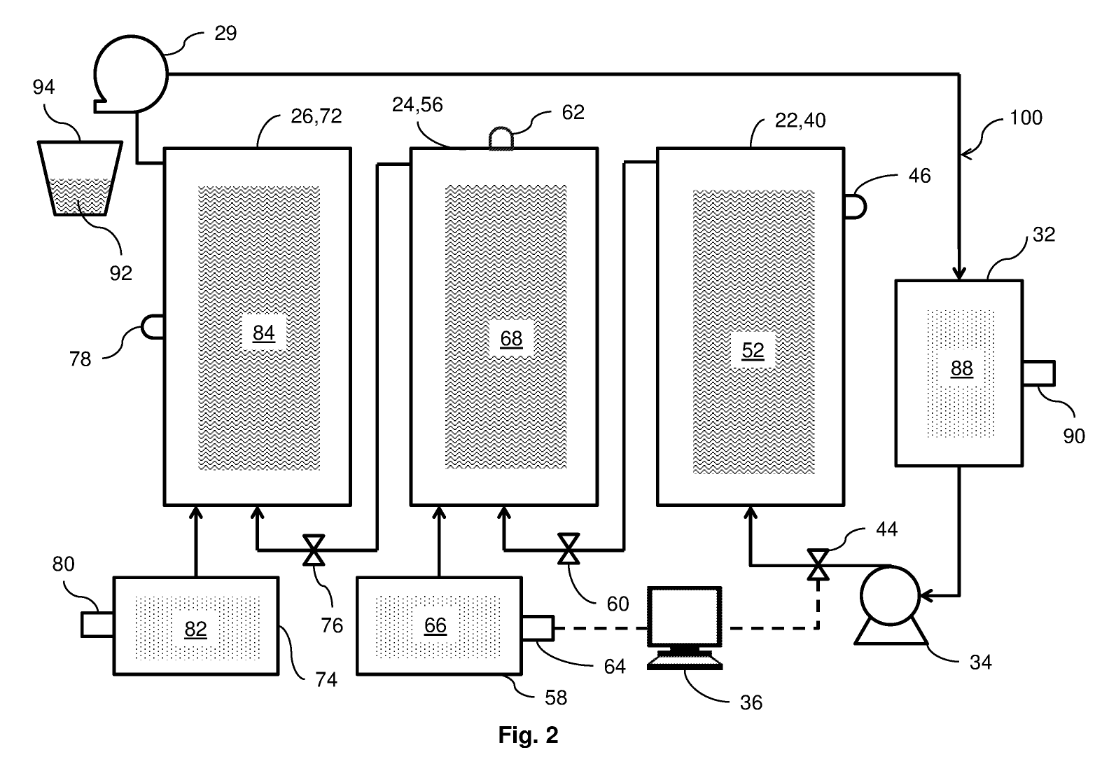

FIG. 2 illustrates a second photobioreactor.

Exemplary, non-limiting embodiments of the present application will now be described with references to the above-mentioned figures. Particularly, FIG. 1 relates to a first embodiment of the present application, whilst FIG. 2 relates to a second embodiment of the present application. Exemplary, non-limiting embodiments of the present application will now be described with references to the above-mentioned figures. Embodiments of the application comprise parts or method steps that are similar or identical to each other. These parts or method steps are thus denoted with similar or identical names or reference numerals. Description of these relevant parts of method steps is hereby incorporated by reference, wherever relevant or appropriate.

According to FIG. 1, the first photobioreactor 20 comprises a first hatcher 22, a second hatcher 24 and a third hatcher 26 that are serially connected together. The first photobioreactor 20 further comprises a control valve 28, a microalgae harvester 29, a filter 30, a first feeder 32, a check valve 33 and a pump 34 that are also sequentially connected to the three hatcheries 20, 22, 24 from the third hatcher 24 to the first hatcher 20. The first photobioreactor 20 additionally comprises a computer 36 that is connected to valves, the pump 34 and sensors of the first photobioreactor 20.

Particularly, the first hatcher 22 comprises a first gas-liquid distributor 38, a first container 40, the first regulating valve 33 (also known as flow regulator or check valve) and the first feeder 32 (also known as the initial feeder) that are joined together. The first container 40 is filled with a first batch of microalgae 52 and further has a first chalcogenide optical fibre biosensor 46 for operating in the mid-infrared (MIR) spectral domain, which is also known as an IR-biosensor 46. Similarly, the first feeder 32 has a first pH sensor 90 that is installed on a wall of the first feeder 32. The first feeder 32 is filled with an initial feedstock 88 (i.e. first feedstock). The initial feedstock is a standard R2 medium comprises of 1.0 g/L of Yeast Extract, 1.0 g/L of Beef Extract, 2.0 g/L of Tryptose, 2.0×10⁻³ g/L of FeSO4, 10.0 g/L of Glucose and 15.0 g/L of Agarose. The first gas-liquid distributor 38 is attached to a bottom of the first container 40, and the first gas-liquid distributor 38 is also directly linked to the pump 34.

Similarly, the second hatcher 24 comprises a second gas-liquid distributor 54, a second container 56, a second feeder 58 and a second regulating valve 60 that are joined together. The second regulator 60 is connected to the computer. The second container 56 has a second IR-biosensor 62. Similarly, the second feeder 58 has a second pH sensor 64 that is installed on a wall of the second container 56. The second feeder 58 is filed with a second feedstock 66, whilst the second container 56 is filled with a second batch of microalgae 68. The second feedstock is a modified R medium comprises of 288 mg/L of Sodium Citrate, 5 mg/L of Ferric Chloride Hexahydrate, 27 mg/L of Calcium Chloride Dihydrate, 488 mg/L Magnesium Sulfate Heptahydrate, 300 mg/L of Ammonia nitrate, 100 mg/L of Potassium Dihydrogen Phosphate, 200 mg/L of Potassium phosphate trihydrate, 900 mg/L of sodium acetate, 5 mg/L of Boric Acid, 5 mg/L of Zinc Sulfate Heptahydrate, 0.15 mg/L of Manganese Sulfate Monohydrate, 0.1 mg/L of Cobalt Chloride Hexahydrate, 0.1 mg/L of Sodium Molybdate Dihydrate, and 0.03 mg/L of Copper Sulfate Pentahydrate. The second gas-liquid distributor 54 is attached to a bottom of the second container 56 and to a top of the first container 40.

Likewise, the third hatcher 26 comprises a third gas-liquid distributor 70, a third container 72, a third feeder 74 and a third regulating valve 76 that are joined together. The third container 72 has a third IR-biosensor 78. Similarly, the third feeder 74 has a third pH sensor 80 that is installed on a wall of the third container 72. The third feeder 74 is filed with a third feedstock 82, whilst the third container 72 is filled with a third batch of microalgae 84. The third feedstock 82 has two times concentration of the modified R medium used in the second feedstock 66, which comprises of 880 mg/L of Sodium Citrate, 10 mg/L of Ferric Chloride Hexahydrate, 53 mg/L of Calcium Chloride Dihydrate, 900 mg/L Magnesium Sulfate Heptahydrate, 600 mg/L of Ammonia nitrate, 200 mg/L of Potassium Dihydrogen Phosphate, 400 mg/L of Potassium phosphate trihydrate, 1800 mg/L of sodium acetate, 10 mg/L of Boric Acid, 10 mg/L of Zinc Sulfate Heptahydrate, 0.3 mg/L of Manganese Sulfate Monohydrate, 0.2 mg/L of Cobalt Chloride Hexahydrate, 0.2 mg/L of Sodium Molybdate Dihydrate, and 0.06 mg/L of Copper Sulfate Pentahydrate. The third gas-liquid distributor 70 is attached to a bottom of the third container 72 and to a top of the second container 56.

An outlet 86 of the third hatcher 26, which is also known as a third outlet 86 of the third container 72, is serially connected to the control valve 28, the microalgae harvester 29, the filter 30, the first feeder 32, the check valve 33, the pump 34 and the first gas-liquid distributor 38. The check valve 33 is also known as a non-return valve or a one-way valve that is configured to allow fluid (liquid or gas) to flow through it in only one direction. The microalgae harvester 29 has a microalgae collector 94 for gathering full-grown microalgae of the photobioreactor 20. The first feeder 32 is filled with an initial feedstock 88, which is also closely monitored by an initial pH sensor 90 on a sidewall of the first feeder 32. The computer 36 is electrically connected to the sensors 46, 90, 62, 64, 78, 80, the valves 28, 33, 33, 60, 76, the pump 34 and other components 29, 30, 38, 54, 70 of the photobioreactor 20 for regulating and monitoring production of the microalgae 52, 68, 84.

Accordingly, the first hatcher 22, the second hatcher 24 and the third hatcher 26 are stacked on top of each other sequentially. The first gas-liquid distributor 38 provides a base 38 of the first photobioreactor 20; the second gas-liquid distributor 54 is joined between the top of the first container 40 and the bottom of the second container 56; and the third gas-liquid distributor 70 is connected between the top of the second container 56 and the bottom of the third container 72. The second feeder 58 and the third feeder 74 are installed at heights higher than their respective containers 56, 72 for allowing natural flow of feedstocks 66, 82 under gravity.

In the photobioreactor 20, the first hatcher 22 (also known as hatchery or incubator) has a closed container 40 with inlets 38 and outlets 54, which facilitates control over the physical, chemical and biological environment of the culture 88 containing the first batch of microalgae 52. Evaporation of the first feedstock 88 at the first container 40, temperature gradients in the first container 40 and protection to the first batch of microalgae 52 from ambient contamination are provided and regulated by the computer 36.

The first container 40 is made of transparent polymethyl methacrylate material supported by frames (not shown). The polymethyl methacrylate material may be replaced by any other one or more types of transparent, translucent or clear materials. The first container 40 has extensive surface areas that are exposed to sunlight and artificial light (e.g. glow plate). Exposure of the first container 40 to the sunlight or the artificial light can be controlled by shades, reflectors and lamps that are attached to the frames. Intensity, wavelength and duration of the light exposure is changed by the shades, reflectors and lamps, which are further organised by the computer 36 for achieving optimised growth of the first batch of microalgae 52. For example, the first container 40 is subjected to artificial illumination with about 120 hours and intensity of 900 μmol/m² sec for growing at 5.8 gram/litre.

The bottom of the first container 40 sits on the first gas-liquid distributor 38. The first gas-liquid distributor 38 has an array of orifices with different sizes and internal channels for guiding flow of bubbles and liquids. Since the first gas-liquid distributor 38 is also connected to the computer 36, sizes of the orifices and channels are regulated for managing carbon dioxide injection and flow rate of the first batch of microalgae 52. For example, the orifices have diameters from 1.2 mm to 2.45 mm, which provide rising of carbon dioxide bubbles in the first container 40 automatically for stirring up the first batch of microalgae 52 in the first container 40, preventing stalemate of the first batch of microalgae 52. Similarly, the second gas-liquid distributor 54 cooperates with the first gas-liquid distributor 38 by exchanging flow of gas and liquid flow in the first container 40. Nutrients and gases are more evenly distributed in the first container 40 by the circulation of fluid between the first gas-liquid distributor 38 and the second gas-liquid distributor 54.

The first pH sensor 90 contacts the first feedstock 88 monitoring pH value change in order to maintain pH value of the first feedstock 88 within a predetermined range. Alarm signals are sent to the computer when detecting abnormal pH values. The first IR-biosensor is adapted to measure photosynthetic activity of the first batch of microalgae 52 by monitoring molecular oxygen production by the microalgae 52 via a luminescent compound, the emission of which depends on the amount of oxygen in the medium. The second regulating valve 60 is electronically controlled by the computer 36. The second regulating valve 60 is configured to open or close fully or partially according to predetermined time periods, which are controlled by the computer 36. The second regulating valve 60 has screens (not shown) that prevent backflow of the first microalgae 52 from the second container 56 to the second feeder 58. The screens, which have passage sizes from a few micrometres (μm) to a few hundreds of micrometres, can be changed or repaired when necessary.

When in use, the first feeder 32, the second feeder 58 and the third feeder 74 are filled feedstocks with increasing values of salinity, nutrients, including inorganic salts, trace elements, and vitamins. The first container 40 is occupied the first batch of microalgae 52 in an aquaculture.

The check valve 33 is opened when the pump 34 starts up by the computer 36. The initial feedstock 88 is propelled by the pump 34 into the first container 40. The initial feedstock 88 is mixed with carbon dioxide gas (CO₂) at the first gas-liquid distributor 38 such that streams of the initial feedstock 88 and bubbles of the carbon dioxide rush into the first container 40, causing gentle turbulence to the aquaculture with the first batch of microalgae 52. Environmental factors of the first hatcher 22, which include temperature, illumination, pH value, CO₂ supply, salt and nutrients levels, are controlled by the computer 36 for achieving optimised growth of the first batch of microalgae 52. The first batch of microalgae 52 has more efficient access to water, carbon dioxide, and other nutrients because the first batch of microalgae 52 grows in aqueous suspension, known as the first feedstock 88.

After about 120 hours, both the second gas-liquid distributor 54 and the second regulating valve 60 opens such that both matured first batch of microalgae 68 and the second feedstock 66 enter the second container 56. The matured first batch of microalgae 68 thus becomes the second batch of microalgae 68. The second gas-liquid distributor 54, similar to the first gas-liquid distributor 38, provides carbon dioxide bubbles and causes gentle flow of the second batch 68. Nutrients in the second container 56, in addition to the sensors 62, 64, are maintained by the second feeder 58, which is also controlled by the computer 36.

In about another 110 hours, both the third gas-liquid distributor 70 and the third regulating valve 76 opens such that both matured second batch of microalgae 84 and the third feedstock 82 flow into the third container 72. The matured second batch of microalgae 84 thus becomes the third batch of microalgae 84. The third gas-liquid distributor 70, similar to the first gas-liquid distributor 38 and the second gas-liquid distributor 54, provides carbon dioxide bubbles and causes gentle circulation of the third batch 88. Nutrients in the third container 72, in addition to the sensors 78, 80, are sustained by the third feeder 82, which is in turn organized by the computer 36.

Full-grown microalgae with their aqueous culture from the third container 72 pass through the third outlet 86 and are received by the microalgae harvester 29. The microalgae harvester 29 employs centrifugation and filtration techniques for obtaining concentrated microalgae in the form of thick algae paste 92, which are collected by a bin 94. In contrast, the feedstock 66, 82, 88 of the aqueous culture is sifted or sieved by the filter 30 and collected by the first feeder 32, at a downstream of the microalgae harvester 29. Filtered aqueous culture is treated as the initial feedstock 88 for feeding into the first container 52 by the check valve 33 and the pump 34.

Referring to FIG. 2, the second photobioreactor 100 comprises a first feeder 32, a pump 34, a first hatcher 22, a second hatcher 24, a third hatcher 26 and a microalgae harvester 29 that are sequentially connected together. These three hatcheries 22, 24, 26 are laid on the ground horizontally such that they 22, 24, 26 have the same height. Broadsides of these hatcheries 22, 24, 26 face top/sky for being exposed to sunlight. The first feeder 32 is linked to the first hatcher 22 via the pump 34 and a first regulating valve 33. The first hatcher 22 is further joined to the second hatcher 24 by a second regulating valve 60, whilst the second hatcher 24 is also coupled to the third hatcher 26 through a third regulating valve 76. Hence, the second photobioreactor 100 is also known as a flat plate photobioreactor or a horizontal photobioreactor.

The first hatcher 22 comprises a first feeder 32, a first IR-biosensor 46 and a first regulating valve 33. The first feeder 32 is connected to a first container 40 of the first hatcher 22 via the pump 34 and the first regulating valve 33 serially. A first pH sensor 90 of the first feeder 32 is installed on a wall of the first feeder 32, whilst a first IP biosensor 46 is mounted onto a wall of the first container 40. A first feedstock 88 and a first batch of microalgae 52 are kept at the first feeder 322 and the first container 40 respectively.

Similarly, the second hatcher 24 comprises a second feeder 58, a second IR-biosensor 62 and a second regulating valve 60. The second feeder 58 is connected to a second container 58 of the second hatcher 24 directly. A second pH sensor 64 of the second feeder 58 is installed on a wall of the second feeder 58, whilst a second IP biosensor 62 is mounted onto a wall of the second container 56. A second feedstock 66 and a second batch of microalgae 68 are kept at the second feeder 58 and the second container 56 respectively.

Likewise, the third hatcher 26 comprises a third feeder 74, a third IR-biosensor 78 and a third regulating valve 76. The third feeder 74 is connected to a third container 72 of the third hatcher 26 directly. A third pH sensor 80 of the third feeder 74 is installed on a wall of the third feeder 74, whilst a third IR-biosensor 78 is mounted onto a wall of the third container 72. A third feedstock 82 and a third batch of microalgae 84 are kept at the third feeder 74 and the third container 72 respectively.

In contrast to the first photobioreactor 20, fluid circulation of the second photobioreactor 100 is achieved peddle wheels and channels in the containers 40, 56, 72. Both the first photobioreactor 20 and the second photobioreactor 100 have immersion heaters (not shown) for maintaining desired temperatures at their respective containers 40, 56, 72. Oxygen and carbon dioxide at the containers 40, 56, 72 are further closely monitored or discharged by the computer 34 when in use.

Hence, the photobioreactors 20, 100 allow multi-stage environments for cultivating microalgae 52, 68, 84. As the initial medium 88 is introduced to the photobioreactors 20, 100 by the liquid pump 34, liquid (aqueous culture) in the first stage 22 together with the microalgae 52 will be “pushed” to the second stage 24. The time of microalgae growth can be controlled by pumping rate of the liquid pump 34. Each stage 22, 24, 26 can have a different volume controlled by the residence time needed at that particular culture environment.

The multi-stage photobioreactors 20, 100 can also be designed in series as shown in FIG. 2. Each stage 22, 24, 26 can be a photobioreactor 22, 24, 26 of same or different structural design. Similarly, the volume of the each photobioreactor 22, 24, 26 is determined by the residence time of the microalgae 52, 68, 84 required at each growth stage 22, 24, 26. The feed medium formula can be independently controlled at each stage of the cultivation.

The photobioreactors 20, 100 can be constructed out of transparent materials, such as glass, acrylic, plastic bag, etc. to allow direct sunlight exposure. It 20, 100 has a multi-compartment design and each compartment 40, 56, 72 may or may not has internal structures to aid circulation. Liquid medium (i.e. aqueous culture) and air/CO₂ gas will be fed from the bottom of the photobioreactors 22, 24, 26. The gas and liquid will pass through the gas-liquid distributors 38, 54, 70 simultaneously while gas will be present in bubble form. The rising bubbles will induce an internal circulation of liquids and prevent settling of the microalgae 52, 68, 84.

The number of stages is preferably between two (02) to fifteen (15). The gas-liquid distributors 38, 54, 70 can introduce gas and liquid mixture as well as allow the pass through of microalgae 52, 68, 84 from one stage to another.

The photobioreactors 20, 100 provide nutrients, growth environment, such as salinity level of the medium, temperature, amount of trace elements, light intensity, etc. that meet changing requirements of the microalgae 52, 68, 84 at different stages of growth process. Therefore, suitable culture environment can be supplied to stacked photobioreactors 22, 24, 26 (reactors or hatcheries) in series to provide the necessary environment for the microalgae 52, 68, 84 at the particular growth stage.

In the application, unless specified otherwise, the terms “comprising”, “comprise”, and grammatical variants thereof, intended to represent “open” or “inclusive” language such that they include recited elements but also permit inclusion of additional, non-explicitly recited elements.

As used herein, the term “about”, in the context of concentrations of components of the formulations, typically means+/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. The description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

It will be apparent that various other modifications and adaptations of the application will be apparent to the person skilled in the art after reading the foregoing disclosure without departing from the spirit and scope of the application and it is intended that all such modifications and adaptations come within the scope of the appended claims.

1. A photobioreactor for cultivating photoautotroph, the photobioreactor comprising:
a first hatcher for containing a first feed medium of the photoautotroph;
a second hatcher for holding a second feed medium of the photoautotroph; and
a valve connected the first hatcher to the second hatcher for regulating flow between the first hatcher and the second hatcher.

2. The photobioreactor of claim 1 further comprising
a third hatcher that is connected to the second hatcher for growing the photoautotrophs progressively in the hatchers with different feed media.

3. The photobioreactor of claim 1 or 2, wherein
at least one of the hatcheries comprises a feeder that is connected to the first hatcher, the second hatcher, the third hatcher or a combination of any of these for providing a feed medium to the photoautotroph.

4. The photobioreactor of claim 3, wherein
the feeder comprises a first feeder connected to the first hatcher for providing the first feed medium.

5. The photobioreactor of claim 3 or 4, wherein
the feeder further comprises a second feeder connected to the second hatcher for providing the second feed medium.

6. The photobioreactor of any of the preceding claims 3 to 5, wherein
the feeder further comprises a third feeder connected to the third hatcher for providing the third feed medium.

7. The photobioreactor of any of the preceding claims further comprising:
a final hatcher additionally connected to the third hatcher for containing a final feed medium of the photoautotroph; and
a final feeder that is connected to the final hatcher for providing the final feed medium.

8. The photobioreactor of claim 1 further comprising
at least one harvest channel that is connected the photobioreactor for harvesting the photoautotroph.

9. The photobioreactor of any of the preceding claims further comprising
a pump for moving the photoautotrophs.

10. The photobioreactor of any of the preceding claims, wherein
at least one of the photobioreactors comprises a gas-liquid distributor for circulating liquid or gas in the at least one photobioreactors.

11. The photobioreactor of any of the preceding claims, wherein
at least two of the hatcheries are stacked.

12. The photobioreactor of any of any of the preceding claims, wherein
at least two of the hatcheries are horizontally laid out.

13. The photobioreactor of any of the preceding claims, wherein
at least two of the hatcheries are different for growing the photoautotrophs differently.

14. The photobioreactor of any of the preceding claims further comprising
at least one sensor for monitoring nutrients in the feed media, growth condition of the photoautotrophs, or components of the photobioreactor.

15. The photobioreactor of claim 14, wherein
the at least one sensor comprises a chemical sensor for monitoring chemical composition of the feed media.

16. The photobioreactor of claim 14 further comprising
the at least one sensor comprising a biosensor for observing growth of the photoautotroph.

17. A cluster of photobioreactors comprising
the photobioreactor according to any of the preceding claims, and
an open pond photobioreactor, a tube photobioreactor, a closed reactor, a horizontal photobioreactor, a vertical photobioreactor, a flat plate reactor, a fermentor-type reactor or a combination of any of these connected to the photobioreactor.

18. A method for cultivating photoautotroph comprising:
providing a first hatcher for cultivating photoautotrophs with a first feed medium;
offering a second hatcher for growing the photoautotrophs with a second feed medium; and
moving the photoautotroph, the first feed medium, the second feed medium or a combination of any of these between the first hatcher and the second hatcher for regulating growth of the photoautotroph.

19. The method of claim 18 comprising:
harvesting the photoautotrophs from the second hatcher that is connected to the first hatcher for progressively growing the photoautotroph.

20. The method of claim 18 or 19 further comprising
propelling the photoautotrophs from the first hatcher to the second hatcher in order to cultivate the photoautotrophs in different media.

21. The method of any of the preceding claims 18 to 20 further comprising
adjusting salinity, temperature, light exposure, nutrient concentration level, oxygen content, carbon dioxide volume, fluid flow rate or other growth environmental factors in at least one of the hatcheries.

22. The method of any of the preceding claims 18 to 21 further comprising
sensing operating condition of the hatcheries.

23. The method of any of the preceding claims 18 to 22 further comprising
collecting growth data of the photoautotrophs via a sensor network via a computer.
